(19) United States
(12) Patent Application Publication
(10) Pub. No.: US 2025/0177915 A1
(43) Pub. Date: Jun. 5, 2025

(54) AIR PURIFIER

(71) Applicant: Samsung Electronics Co., Ltd., Suwon-si (KR)

(72) Inventors: Jaiyoung CHUNG, Suwon-si (KR); Dongwook KIM, Suwon-si (KR); Joonseon JEONG, Suwon-si (KR); Su Keun KUK, Suwon-si (KR); Hyun Chul LEE, Suwon-si (KR)

(21) Appl. No.: 18/827,199

(22) Filed: Sep. 6, 2024

(30) Foreign Application Priority Data
Dec. 1, 2023 (KR) 10-2023-0172736

(51) Int. Cl. B01D 53/32; A61L 9/22; B01D 53/88

(52) U.S. Cl. CPC B01D 53/32; A61L 9/22; B01D 53/885; B01D 2258/06; B01D 2259/818

(57) ABSTRACT

An air purifier includes a reactor including a hollow region extending in a first direction, a first electrode unit extending in the first direction and disposed in the hollow region, a plurality of supports disposed in the hollow region to surround the first electrode unit, and spaced apart from each other in the first direction, where a plurality of through holes extending in the first direction is defined in each of the plurality of supports, a plurality of discharge elements coupled to the first electrode unit and disposed in the hollow region, where at least one of the plurality of discharge elements is disposed in a first discharge unit defined by a space between the plurality of supports, and a second electrode unit spaced apart from the plurality of discharge elements by a certain distance on a plane perpendicular to the first direction to surround the plurality of discharge elements.

### CROSS-REFERENCE TO RELATED APPLICATION

This application claims priority to Korean Patent Application No. 10-2023-0172736, filed on Dec. 1, 2023, and all the benefits accruing therefrom under 35 U.S.C. § 119, the content of which in its entirety is herein incorporated by reference.

### BACKGROUND

### 1. Field

The disclosure relates to an air purifier for purifying a pollutant gas containing a pollutant.

### 2. Description of the Related Art

An air purifier purifies air by collecting or decomposing gas, for example, fine dust and pollutants in the air. The air purifier may be used in industrial dust collection equipment, an air conditioning/ventilating system in a building, etc.

Recently, as the legal regulations for the atmospheric environment have become more stringent, techniques for treating odorants such as ammonia, hydrogen sulfide, etc., and volatile organic compounds (VOCs) such as toluene and xylene have been actively developed.

Plasma, especially, low-temperature plasma may provide a region of high reactivity at a room temperature by generating various active species such as electrons, ions, radicals, etc. of high energy at a temperature close to the room temperature. In a region of high reactivity using plasma, pollutants including a volatile organic compound may be purified. When plasma and a catalyst are combined, active species generated in the plasma may act on the catalyst, and thus, a volatile organic compound decomposition catalyst generally working at high temperatures of 200 degrees or more may operate at the room temperature.

### SUMMARY

Embodiments relate to an air purifier in which an active specie generated in plasma acts on a catalyst.

Embodiments relate to an air purifier with increased activation efficiency of a catalyst.

According to an embodiment of the disclosure, an air purifier includes a reactor including a hollow region extending in a first direction, a first electrode unit extending in the first direction and disposed in the hollow region, a plurality of supports disposed in the hollow region to surround the first electrode unit, and spaced apart from each other in the first direction, where a plurality of through holes extending in the first direction is defined in each of the plurality of supports, a plurality of discharge elements coupled to the first electrode unit and disposed in the hollow region, where at least one of the plurality of discharge elements is disposed in a first discharge unit defined by a space between the plurality of supports, and a second electrode unit spaced apart from the plurality of discharge elements by a certain distance on a plane perpendicular to the first direction to surround the plurality of discharge elements.

In an embodiment, each of the plurality of supports may include a catalyst coated along a surface thereof.

In an embodiment, one of the plurality of discharge elements may be disposed between two adjacent supports among the plurality of supports.

In an embodiment, the two adjacent supports may fix a coupling position of the one of the plurality of discharge elements disposed therebetween on the first electrode unit.

In an embodiment, the plurality of supports and the plurality of discharge elements may be alternately disposed in the first direction.

In an embodiment, the plurality of supports and the plurality of discharge elements may alternately contact each other in the first direction.

In an embodiment, the reactor may include a dielectric material.

In an embodiment, the second electrode unit may surround the reactor.

In an embodiment, the discharge element may include a protrusion pattern protruding towards the second electrode unit.

In an embodiment, each of the plurality of supports may have a honeycomb structure.

In an embodiment, the plurality of discharge elements and the plurality of supports may form a discharge region within the reactor, and one of the plurality of discharge elements may be disposed in a second discharge unit defined by both opposing ends of the discharge region.

In an embodiment, the one of the plurality of discharge elements disposed in the second discharge unit may contact an adjacent support.

In an embodiment, a position of the one of the plurality of discharge elements disposed in the second discharge unit may be fixed to the first electrode unit.

In an embodiment, the air purifier may further include an external catalyst surrounding the first electrode unit and disposed outside the discharge region, and the external catalyst may be disposed at a rear end of the discharge region.

In an embodiment, the external catalyst may be disposed adjacent to the second discharge unit.

In an embodiment, a distance between the external catalyst and the second discharge unit may be 120% or less of a distance between the reactor and the discharge element disposed in the second discharge unit.

In an embodiment, the distance between the external catalyst and the second discharge unit may be 100% or less of the distance between the reactor and the discharge element disposed in the second discharge unit.

In an embodiment, the reactor may have a cross-section of a circular shape or a polygonal shape.

In an embodiment, the reactor, the first electrode unit, the plurality of supports, the plurality of discharge elements, and the second electrode unit may collectively define a plasma reaction module, and the plasma reaction module may be provided in plural, and a plurality of reactors respectively included in a plurality of plasma reaction modules are disposed to be adjacent to each other.

In an embodiment, the air purifier may further include a cooling flow path disposed between the plurality of reactors respectively included in the plurality of plasma reaction modules, and cooling water provided to move along the cooling flow path.

### BRIEF DESCRIPTION OF THE DRAWINGS

The above and other features of certain embodiments of the disclosure will be more apparent from the following description taken in conjunction with the accompanying drawings, in which:

FIG. 1 is a perspective view of an air purifier according to an embodiment;

FIG. 2 is a block diagram of the air purifier according to an embodiment;

FIG. 3 is a cross-sectional view of the air purifier shown in FIG. 1 viewed in a direction A-A′;

FIG. 4 is an enlarged view of a region M shown in FIG. 3;

FIG. 5 is a cross-sectional view showing a discharge element of the air purifier according to an embodiment;

FIG. 6 is a cross-sectional view of the air purifier according to an embodiment;

FIG. 7 is a perspective view of the air purifier further including an external catalyst according to an embodiment;

FIG. 8 is a cross-sectional view of the air purifier further including the external catalyst according to an embodiment;

FIG. 9A is a front view of a plasma reaction module according to an embodiment;

FIG. 9B is a front view of a plasma reaction module according to an embodiment;

FIG. 9C is a front view of a plasma reaction module according to an embodiment;

FIG. 9D is a front view of a plasma reaction module according to an embodiment;

FIG. 10A is a perspective view of an air purifier including a plurality of plasma reaction modules according to an embodiment;

FIG. 10B is a front view of the air purifier including the plurality of plasma reaction modules according to an embodiment;

FIG. 11A is a perspective view of an air purifier including a cooling flow path according to an embodiment; and

FIG. 11B is a front view of the air purifier including the cooling flow path according to an embodiment.

### DETAILED DESCRIPTION

The invention now will be described more fully hereinafter with reference to the accompanying drawings, in which various embodiments are shown. This invention may, however, be embodied in many different forms, and should not be construed as limited to the embodiments set forth herein. Rather, these embodiments are provided so that this disclosure will be thorough and complete, and will fully convey the scope of the invention to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present.

It will be understood that, although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein

The terminology used herein is for the purpose of describing particular embodiments only and is not intended to be limiting. As used herein, “a”, “an,” “the,” and “at least one” do not denote a limitation of quantity, and are intended to include both the singular and plural, unless the context clearly indicates otherwise. Thus, reference to “an” element in a claim followed by reference to “the” element is inclusive of one element and a plurality of the elements. For example, “an element” has the same meaning as “at least one element,” unless the context clearly indicates otherwise. “At least one” is not to be construed as limiting “a” or “an.” “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Also, the terms “ . . . unit” and “ . . . module” used herein specify a unit for processing at least one function or operation, and this may be implemented with hardware or software or a combination of hardware and software. The connection or connection members of the lines between the components shown in the drawing exemplarily represent functional and/or physical or circuit connections; in real devices, they may be represented as replaceable or additional various functional connections, physical connections, or circuit connections. The use of all examples or exemplary terms provided herein, are intended merely to better illuminate the technical ideas and does not pose a limitation on the scope of rights unless otherwise claimed.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The terms “below” or “beneath” can, therefore, encompass both an orientation of above and below.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +30%, 20%, 10% or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

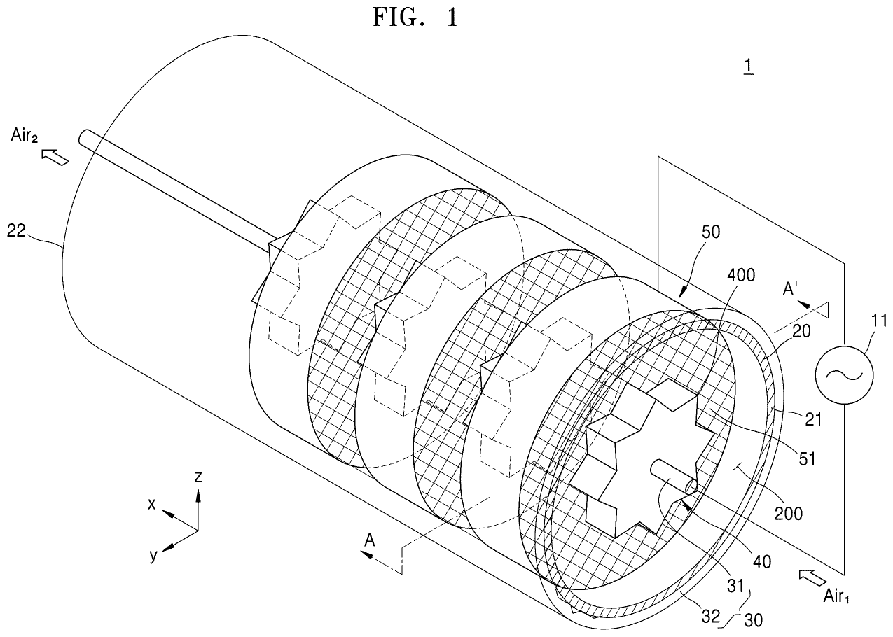

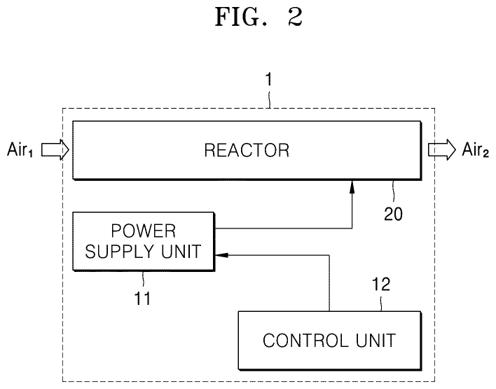

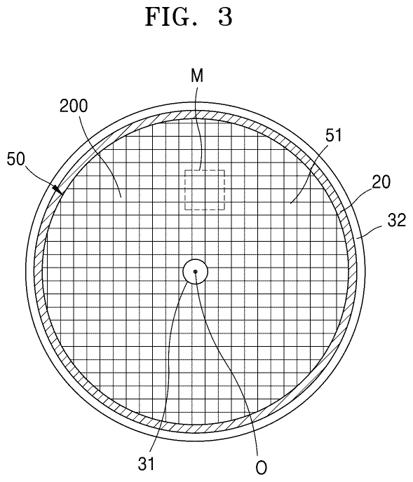

FIG. 1 is a perspective view of an air purifier 1 according to an embodiment. FIG. 2 is a block diagram of the air purifier 1 according to an embodiment. FIG. 3 is a cross-sectional view of the air purifier 1 shown in FIG. 1 viewed in a direction A-A′.

Referring to FIGS. 1 to 3, the air purifier 1 according to an embodiment may be configured to purify polluted air Air₁. The air purifier 1 may be configured to allow the polluted air Air₁ to be introduced therein. The air purifier 1 may be configured to purify the polluted air Air₁ into purified air Air₂. The air purifier 1 may purify the polluted air Air₁ introduced thereinto and discharge the purified air Air₂.

The polluted air Air₁ may mean a mixed gas including air and one or more of a particulate matter, a water-soluble organic compound, and a water-insoluble organic compound. For example, the particulate matter may include a small particulate matter of about 10 micrometers (μm) or less and ultrafine dust of about 2.5 μm or less. In addition, the water-soluble organic compound, which is a volatile organic compound, may include a gas material removable by being collected in water or an aqueous solution, e.g., ammonia (NH₃), acetaldehyde (CH₃CHO), and acetic acid (CH₃COOH). The water-insoluble organic compound, which is a volatile organic compound not collected in water or an aqueous solution, may include, for example, benzene (C₆H₆), formaldehyde (CH₂O), toluene (C₆H₅CH₃), etc. However, the disclosure is not limited thereto, and other random gases than the particulate matter, water-soluble organic compound, and the water-insoluble organic compound may be included in the polluted air Air₁.

Air purifier 1 according to an embodiment may include a reactor 20. The reactor 20 may provide a space in which the polluted air Air₁ is purified into the purified air Air₂. Inside the reactor 20, the polluted air Air₁ may be purified into the purified air Air₂. External air may be introduced into the reactor 20. The polluted air Air₁ may be introduced into the reactor 20. Inside the reactor 20, the polluted air Air₁ may be purified into the purified air Air₂. The reactor 20 may discharge the purified air Air₂ to the outside.

The reactor 20 according to an embodiment may provide a flow path of air. The reactor 20 may provide the flow path through which air flows in a process of purifying the polluted air Air₁ introduced from the outside into the purified air Air₂. In other words, the flow path through which air flows into the reactor 20 may be formed in the reactor 20.

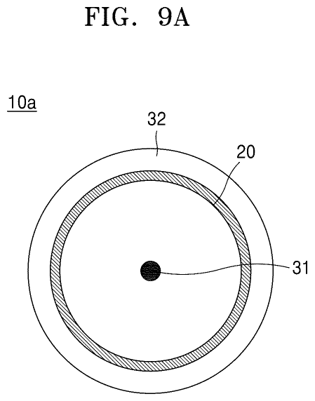

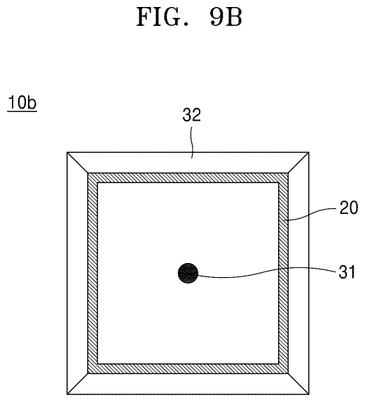

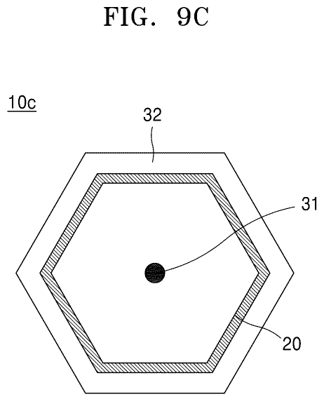

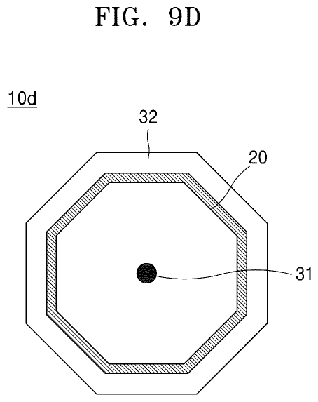

The reactor 20 according to an embodiment may extend in a first direction x. The first direction x may indicate a direction in which the reactor 20 extends. The first direction x may indicate a direction in which air flows inside the reactor 20. The reactor 20 may have a hollow region 200. The hollow region 200 of the reactor 20 may be a space in which the polluted air Air₁ flows. The hollow region 200 of the reactor 20 may extend in the first direction x. The polluted air Air₁ may flow through the hollow region 200 provided by the reactor 20. Inside the hollow region 200 of the reactor 20, the polluted air Air₁ may be purified into the purified air Air₂. However, the above description of the function of the reactor 20 and the flow of air within the hollow region 200 is only an example and the invention is not limited thereto. The reactor 20 according to an embodiment may have a conduit shape. The reactor 20 may have a substantially cylindrical shape. The reactor 20 may have a circular cross-sectional shape with respect to the first direction x. However, the shape of the reactor 20 is not limited to the above description, and may have various shapes as long as the reactor 20 may provide the hollow region 200 through which air may flow. In an embodiment, for example, the reactor 20 may have a polygonal cross-sectional shape with respect to the first direction x. Examples of various cross-sectional shapes that the reactor 20 may have are described below based on a cross-sectional shape of a plasma reaction module with reference to FIGS. 9A to 9D.

Although not shown, the air purifier 1 may include a pressure application device. The pressure application device may generate a suction pressure so that the polluted air Air₁ is introduced into the air purifier 1. The pressure application device may generate the suction pressure so that air may flow inside the air purifier 1. In other words, the pressure application device may apply a negative pressure below (lower than) the atmospheric pressure to the reactor 20. The pressure application device may be configured to discharge the purified air Air₂ inside the air purifier 1 to the outside of the air purifier 1. In an embodiment, for example, the pressure application device may include a fan or pump. In an embodiment, for example, the pressure application device may be disposed at a rear end of the reactor 20. However, the description of the type and arrangement of the pressure application device is only an illustrative description and the invention is not limited thereto.

The reactor 20 according to an embodiment may include a dielectric material. In an embodiment, for example, the reactor 20 may be provided as a ceramic conduit extending in the first direction x. In an embodiment, for example, the reactor 20 may be provided as a metal conduit extending in the first direction x. However, the material and shape of the reactor 20 are not limited thereto and the reactor 20 may have various materials and shapes as long as plasma, which will be described below, is allowed to be generated in the hollow region 200 inside the reactor 20.

The air purifier 1 according to an embodiment may include a discharge electrode 30. The discharge electrode 30 may discharge plasma to a discharge region 60 inside the air purifier 1. The discharge electrode 30 may discharge plasma to the discharge region 60 inside the reactor 20. The discharge electrode 30 may purify the polluted air Air₁ into the purified air Air₂ by discharging plasma to the discharge region 60.

The discharge electrode 30 according to an embodiment may include a first electrode unit 31. The first electrode unit 31 may have a rod shape. The first electrode unit 31 may extend in a direction in which the reactor 20 extends. The first electrode unit 31 may extend along a flow path through which air flows inside the air purifier 1. The first electrode unit 31 may extend in the first direction x. The first electrode unit 31 may have a certain cross-sectional shape along a plane perpendicular to the first direction x. In an embodiment, for example, the first electrode unit 31 may have either a circular or polygonal cross-sectional shape.

The first electrode unit 31 according to an embodiment may be disposed inside the air purifier 1. The first electrode unit 31 may be disposed inside the reactor 20. The first electrode unit 31 may be disposed in the hollow region 200 of the reactor 20. In an embodiment, as shown in FIG. 3, the first electrode unit 31 may be disposed at a center O of the hollow region 200. In such an embodiment where the first electrode unit 31 is disposed at the center O of the hollow region 200, a relatively uniform voltage may be formed in the discharge electrode 30. However, the shape and arrangement of the first electrode unit 31 are not limited to the above description.

The first electrode unit 31 may include a metal material extending in the first direction x. In an embodiment, for example, the first electrode unit 31 may be provided as a stainless (SUS) wire extending in the first direction x and may be disposed in the hollow region 200 of the reactor 20. In an embodiment, for example, a length of the first electrode unit 31 extending in the first direction x may be about 1 millimeter (mm) or greater and about 1500 mm or less. However, the material of the first electrode unit 31 is not limited thereto, and the first electrode unit 31 may be connected to a power supply unit 11, which will be described below, to have an arbitrary electrode structure for forming a high voltage in the discharge electrode 30.

The discharge electrode 30 according to an embodiment may include a second electrode unit 32. The second electrode unit 32 may be a ground electrode. The second electrode unit 32 may surround the first electrode unit 31. The second electrode unit 32 may be spaced apart from the first electrode unit 31 with a certain gap therebetween.

The second electrode unit 32 according to an embodiment may surround the reactor 20. The second electrode unit 32 may extend in the first direction x. The second electrode unit 32 may surround the reactor 20 and extend in the first direction x. in an embodiment, where the reactor 20 is a non-conductor, the second electrode unit 32 may surround an outer wall of the reactor 20. In an embodiment, for example, where the reactor 20 is a ceramic conduit, the second electrode unit 32 may be provided as (or defined by) an aluminum thin film provided to surround the outer wall of the reactor 20. The second electrode unit 32 may be integrated (or integrally formed) with the reactor 20 in an embodiment where the reactor 20 is a conductor. In an embodiment, for example, where the reactor 20 is an aluminum conduit, the second electrode unit 32 may be integrated with the reactor 20 and redisposed with the reactor 20. However, the description of the arrangement and materials of the second electrode unit 32 and the reactor 20 is only an example and the invention is not limited thereto.

The air purifier 1 according to an embodiment may include the power supply unit 11 configured to apply a voltage to the discharge electrode 30. The power supply unit 11 may form an electric field inside the reactor 20 by applying a voltage to the discharge electrode 30. The power supply unit 11 may form the electric field on the hollow region 200. The power supply unit 11 may apply a high voltage to the discharge region 60 where a discharge plasma may be generated. In an embodiment, for example, the power supply unit 11 may apply a certain voltage between the first electrode unit 31 and the second electrode unit 32 to generate a plasma between the first electrode unit 31 and the second electrode unit 32. In an embodiment, for example, the power unit 11 may include a sinusoidal alternating current (AC) power supply and a transformer. The power supply unit 11 may continuously apply the high voltage to the inside of the reactor 20, for example, to the discharge region 60 where the discharge plasma may be generated through the above-described electrical system. In an embodiment, for example, the voltage applied to the discharge region 60 inside the reactor 20 may be about 0.5 kilovolt (kV) or greater and about 500 KV or less, and a frequency may be about 10 hertz (Hz) or greater and about 100 GHz or less, but the disclosure is not limited thereto. In an embodiment, for example, a direct current voltage may be applied to the inside of the discharge region 60. In addition, a separation distance between the first electrode unit 31 and the second electrode unit 32 in the discharge region 60 may be about 10 mm or greater and about 100 mm or less, and accordingly, an electric field of about 2 kilovolt per centimeter (kV/cm) or greater and about 5 kV/cm or less may be applied to the discharge region 60. However, the voltage applied by the power supply unit 11 to the first electrode unit 31 and the second electrode unit 32 and the electric field formed in the discharge region 60 inside the reactor 20 are not limited to the above description. The air purifier 1 according to an embodiment may further include a control unit 11 configured to control an operation (e.g., voltage generations) of the power supply unit 11.

The first electrode unit 31 and the second electrode unit 32 according to an embodiment may form a plasma in the discharge region 60. The plasma formed in the discharge region 60 may purify the polluted air Air₁. The polluted air Air₁ may be purified by the plasma formed in the discharge region 60 inside a support 50. The air purifier 1 may purify the polluted air Air₁ with the plasma and discharge the purified air Air₂.

The air purifier 1 according to an embodiment may include the support 50. The support 50 may surround the first electrode unit 31. The support 50 may be disposed between the first electrode unit 31 and the second electrode unit 32. The support 50 may be disposed inside the reactor 20. The support 50 may be disposed in the hollow region 200 of the reactor 20.

The support 50 according to an embodiment may be configured to support the reactor 20. The support 50 may contact an inner surface of the reactor 20. The support 50 may support the reactor 20 inside the reactor 20. The support 50 may support the reactor 20 so that the reactor 20 and the first electrode unit 31 maintain a certain gap.

The support 50 may support the first electrode unit 31 and the reactor 20 so that the first electrode unit 31 may be located in an inner central portion of the reactor 20. However, the arrangement and function of the support 50 are not limited to the above description.

In an embodiment, the support 50 may include a dielectric material having a certain dielectric constant. In an embodiment, for example, the support 50 may include at least one selected from metal oxide, metal nitride, and high molecular weight polymer. According to an embodiment, where the reactor 20 is implemented as a non-conductor, for example, a ceramic conduit, the support 50 including ceramic may be formed integrally with the reactor 20 as a single unitary indivisible part. However, the disclosure is not limited thereto, and in an embodiment where the reactor 20 is implemented as a conductor, for example, an aluminum conduit or includes a material different from the support 50, the reactor 20 and the support 50 may not be formed integrally with each other but provided as separate structures.

The support 50 according to an embodiment may be provided with a plurality of through holes 51 defined therein to extend in the first direction x. In an embodiment, for example, the plurality of through holes 51 may extend from a front end of the reactor 20 to a rear end of the reactor 20. Accordingly, a plurality of flow paths extending from the front end of the reactor 20 to the rear end of the reactor 20 may be formed. The front end of the reactor 20 may be one end of the reactor 20 into which the polluted air Air₁ is introduced. The rear end of the reactor 20 may be the other end of the reactor 20 from which the purified air Air₂ is discharged. In an embodiment, for example, the polluted air Air₁ introduced into the front end of the reactor 20 may be moved to the rear end of the reactor 20 through the plurality of through holes 51.

The plurality of through holes 51 according to an embodiment may provide a flow path through which air flows within the reactor 20. The polluted air Air₁ introduced into the reactor 20 may flow along the plurality of through holes 51. The polluted air Air₁ introduced into the reactor 20 may flow in the first direction x along the plurality of through holes 51 and a pollutant included in the polluted air Air₁ may be removed. When the polluted air Air₁ flows along the plurality of through holes 51 inside the support 50, the pollutant may be removed by a chemical reaction induced by a catalyst.

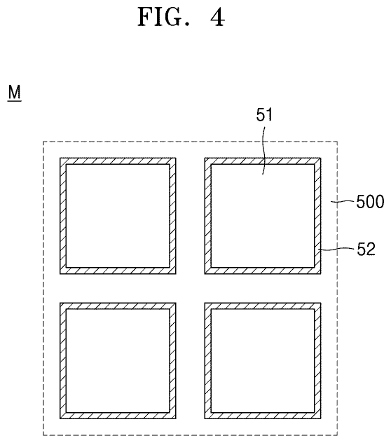

Hereinafter, a catalyst 52 coated along a surface of the support 50 will be described. FIG. 4 is an enlarged view of a region M shown in FIG. 3.

Referring to FIGS. 1, 3, and 4, the air purifier 1 according to an embodiment may include a catalyst 52. The catalyst 52 may remove a pollutant contained in the polluted air Air₁. The catalyst 52 may be coated along the surface of the support 50. The catalyst 52 may be coated along inner surfaces of the support 50 defining the plurality of through holes 51. The catalyst 52 may be in contact with the polluted air Air₁ flowing within the plurality of through holes 51. In an embodiment, for example, the catalyst 52 may include at least one selected from Ti, Al, Zn, Cu, Mg, Si, Ni, Pt, Rh, Au, Ru, and an oxide thereof. However, a type of catalyst 52 is not limited thereto and the catalyst 52 may include an arbitrary material capable of removing the pollutant contained in the polluted air Air₁.

The support 50 according to an embodiment is provided with the plurality of through holes 51, and thus, an area where the polluted air Air₁ contacts the catalyst 52 may be large. The plurality of through holes 51 may increase the contact area between the catalyst 52 and the polluted air Air₁. When the catalyst 52 contacts the polluted air Air₁ on a large area, air purification efficiency may be high. The plurality of through holes 51 may increase the air purification efficiency of the catalyst 52.

As the support 50 according to the embodiment is provided with the plurality of through holes 51, a pressure difference between a front end of the reactor 20 and a rear end of the reactor 20, that is, a differential pressure between the front end of the reactor 20 and the rear end of the reactor 20 may be reduced. In a case, for example, where a dielectric material in the form of pellet or powder is filled in the reactor 20, a diameter of a flow path through which the polluted air Air₁ may pass may not be sufficiently secured. In addition, in such a case where the dielectric material in the form of pellet or powder is filled in the reactor 20, the flow path is provided in a curved shape rather than a straight line, and thus, a length of the flow path through which the polluted air Air₁ passes may increase. Therefore, when the polluted air Air₁ of a same flow rate is introduced to the front end of the reactor 20, differential pressure between the front end of the reactor 20 and the rear end of the reactor 20 may be more reduced in a case where the plurality of through holes 51 are defined in the discharge region 60 of the reactor 20 than a case the dielectric material in the form of pallet or powder is filled in the reactor 20. When the differential pressure between the front end of the reactor 20 and the rear end of the reactor 20 is reduced, the flow rate of air that may flow into the air purifier 1 may be large. When the differential pressure between the front end of the reactor 20 and the rear end of the reactor 20 is reduced, the flow rate of polluted air Air₁ that the air purifier 1 may purify may be large. In an embodiment, for example, the differential pressure between the front end of the reactor 20 and the rear end of the reactor 20 may be about 10 pascal (Pa) or greater and about 1000 Pa or less. However, the above description of the reduction in the differential pressure between the front end of the reactor 20 and the rear end of the reactor 20 due to the plurality of through holes 51 is only an illustrative description and is not limited thereto. The structure and function of the plurality of through holes 51 may be set differently according to the purpose of use of the air purifier 1.

The support 50 according to an embodiment may have a honeycomb structure. The support 50 may have the honeycomb structure in which the plurality of through holes 51 extending in the first direction x are defined. In an embodiment, for example, cell density of catalyst pores of the support 50 having the honeycomb structure may be about 40 cells per square inch (cpsi) or greater and about 1800 cpsi or less. A thickness of the support 50 having the honeycomb structure may be about 1 mm or greater and about 2000 mm or less. The cross-sectional shape of the plurality of through holes 51 along a plane perpendicular to the first direction x may include one or more of a polygonal shape and a circular shape. In an embodiment, for example, the plurality of through holes 51 may have a rectangular cross-section. However, the cross-sectional shape of the plurality of through holes 51 is not limited thereto, and the plurality of through holes 51 may have an arbitrary cross-sectional shape through which the polluted air Air₁ may pass.

The catalyst 52 according to an embodiment may be combined with a plasma generated by the discharge electrode 30. In an embodiment, the catalyst 52 may be combined with an active specie generated in the plasma. The active specie generated in the plasma may act on the catalyst 52. When the active specie generated in the plasma acts on the catalyst 52, a volatile organic compound decomposition catalyst which acts at a high temperature of 200 degrees or higher may act even at a room temperature. The room temperature may be a temperature in a range of about 1° C. to about 35° C. (e.g., in a range of about 20° C. to about 25° C.). Accordingly, the catalyst 52 may decompose volatile organic compound with high efficiency.

In an embodiment, the time for which the plasma remains in the reactor 20 is very short, ranging from nanoseconds to microseconds. Accordingly, the time for which the catalyst 52 may interact with the plasma may also be very short, ranging from nanoseconds to microseconds. If the catalyst 52 is disposed far from a position where the plasma is generated, it may be difficult for the plasma to act on the catalyst 52.

The support 50 according to an embodiment may be disposed adjacent to a region where the plasma is generated within the reactor 20. In an embodiment, for example, the support 50 may be disposed adjacent to the discharge region 60 configured to generate the plasma between the first electrode unit 31 and the second electrode unit 32. As described above, the catalyst 52 may be coated along a surface of the support 50. When the support 50 is disposed adjacent to the discharge region 60 inside the reactor 20, a distance of a movement of the plasma to act on the catalyst 52 may be short. When the support 50 is disposed adjacent to the discharge region 60 inside the reactor 20, the plasma may well act on the catalyst 52. When the support 50 is disposed adjacent to the discharge region 60 inside the reactor 20, the activation efficiency of the catalyst 52 by the plasma may be high. When the support 50 is disposed adjacent to the discharge region 60 inside the reactor 20, the air purification efficiency of the air purifier 1 may be high.

In an embodiment, a region where the air purifier 1 forms the plasma inside the reactor 20 may be concentrated in a specific region. The discharge region 60 where the plasma is formed inside the reactor 20 may be concentrated in a region where a discharge element 40, which will be described below, is disposed. Hereinafter, the discharge element 40 included in the air purifier 1 will be described.

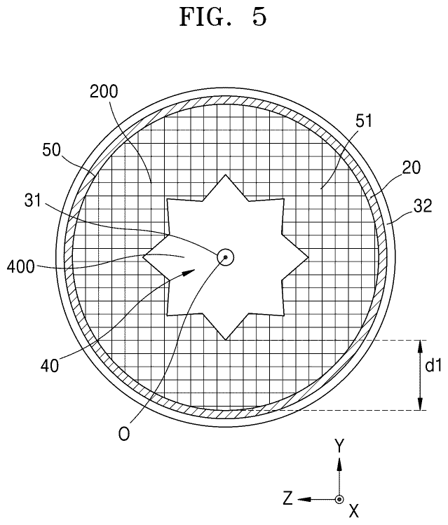

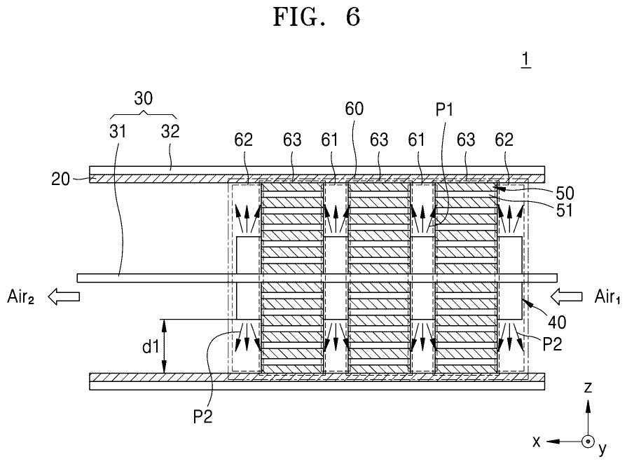

FIG. 5 is a cross-sectional view showing the discharge element 40 of the air purifier 1 according to an embodiment. FIG. 6 is a cross-sectional view of the air purifier 1 according to an embodiment.

Referring to FIGS. 1 to 6, the air purifier 1 according to an embodiment may include the discharge element 40. The discharge element 40 may form a region where plasma is intensively generated inside the reactor 20. The discharge element 40 may be disposed inside the reactor 20. The discharge element 40 may be disposed in the hollow region 200 of the reactor 20. The discharge element 40 may be electrically connected to the first electrode unit 31. The discharge element 40 may receive power from the power supply unit 11 through the first electrode unit 31. The power supply unit 11 may supply the power to the discharge element 40 through the first electrode unit 31. A material of the discharge element 40 may be the same as a material of the first electrode unit 31, but is not limited thereto. The discharge element 40 may include various materials as long as the discharge element 40 is electrically connected to the first electrode unit 31 and receives the power supplied by the power supply unit 11.

The discharge element 40 according to an embodiment may be disposed to surround the first electrode unit 31. The discharge element 40 being disposed to surround the first electrode unit 31 may mean that the discharge element 40 may surround at least a part of a circumference of the first electrode unit 31 and extend in the first direction x. In other words, the discharge element 40 may cover at least a part of the first electrode unit 31. A region where the discharge element 40 covers the first electrode unit 31 may be referred to as a first discharge unit 61 and a second discharge unit 62, which will be described below.

The discharge element 40 may be spaced apart from the second electrode unit 32 by a certain distance. The second electrode unit 32 may be disposed to surround the discharge element 40. The discharge element 40 may be disposed at the center O of the hollow region 200 inside the reactor 20. The discharge element 40 may be disposed at the center O of the hollow region 200. In an embodiment where the discharge element 40 is disposed at the center O of the hollow region 200, a uniform voltage may be formed with the second electrode unit 32. The discharge element 40 may be spaced apart by a certain gap inside the reactor 20. The reactor 20 may surround the discharge element 40. A separation distance between the discharge element 40 and the reactor 20 may be a first distance d1, which will be described in detail below.

The discharge element 40 may be coupled to the first electrode unit 31. The discharge element 40 being coupled to the first electrode unit 31 may mean that the first electrode unit 31 may penetrate a coupling hole defined or formed in the center O of the discharge element 40. The discharge element 40 being coupled to the first electrode unit 31 may mean that the discharge element 40 may move in parallel in the first direction x and be inserted into the first electrode unit 31. The discharge element 40 being coupled to the first electrode unit 31 may mean that the discharge element 40 moves in parallel in the first direction x within the reactor 20 while the first electrode unit 31 penetrates the discharge element 40, and may be disposed at a predetermined position. The discharge element 40 being coupled to the first electrode unit 31 may mean that a position of the discharge element 40 is fixed while the discharge element 40 is disposed at an intended position within the reactor 20. When the discharge element 40 is disposed at the intended location within the reactor 20, a region where plasma is intensively formed within the reactor 20 may be appropriately designed. Fixing the position of the discharge element 40 may mean fixing a relative position between the first electrode unit 31 and the discharge element 40. However, a method of coupling the discharge element 40 to the first electrode unit 31 is not limited to the above description. In an embodiment, for example, the discharge element 40 may be formed integrally with the first electrode unit 31 as a single unitary indivisible part.

At least a part of the second electrode unit 32 according to an embodiment may surround the plurality of discharge elements 40. Here, the second electrode unit 32 surrounding the discharge elements 40 may mean that the second electrode unit 32 may surround the discharge element 40 at a position spaced apart by a certain distance in a direction perpendicular to the first direction x from a position where the discharge element 40 is disposed. In other words, with respect to the cross-section of the discharge element 40 extending in the first direction x, a cross-section of at least a part of the second electrode unit 32 may surround the discharge element 40. More specifically, referring to FIG. 5, the cross-section of at least a part of the second electrode unit 32 may surround the cross-section of the discharge element 40 extending in the first direction x.

At least a part of the second electrode unit 32 according to an embodiment may be disposed to surround the discharge element 40 on a plane perpendicular to the first direction x. For example, the plane perpendicular to the first direction x may be an yz plane. For example, the plane perpendicular to the first direction x may be parallel to a cross-section along which the reactor 20 extends. At least a part of the second electrode unit 32 may be spaced apart from the plurality of discharge elements 40 by a certain distance on the plane perpendicular to the first direction x to surround the plurality of discharge elements 40. The reactor 20 may be disposed between the second electrode unit 32 and the discharge element 40. The second electrode unit 32 may be disposed to surround the outside of the reactor 20 surrounding the discharge element 40 on the plane perpendicular to the first direction x. In other words, the reactor 20 may be disposed to surround the discharge element 40 on the plane perpendicular to the first direction x, and the second electrode unit 32 may be disposed to surround the reactor 20. However, an arrangement relationship between the discharge element 40 and the second electrode unit 32 is not limited to the above description.

A distance between the discharge element 40 and the second electrode unit 32 according to an embodiment may be shorter than a distance between the first electrode unit 31 and the second electrode unit 32. The discharge element 40 may generate a plasma in the direction towards the second electrode unit 32 as the power supply unit 11 applies a high voltage to the discharge electrode 30. As the distance between the discharge element 40 and the second electrode unit 32 is shorter than the distance between the first electrode unit 31 and the second electrode unit 32, a plasma generation may be concentrated in the discharge element 40. However, when the plasma is generated in the discharge region 60, the plasma may also be generated in the first electrode unit 31 where the discharge element 40 is not disposed. In an embodiment, for example, as shown in FIG. 6, the discharge element 40 may not be disposed in the region where a support portion 63 is disposed, but the plasma may be generated between the first electrode unit 31 and the second electrode unit 32 even in the region where the support portion 63 is disposed. In other words, the discharge element 40 may serve to induce the formation of a region where the plasma is intensively generated in at least a part of the discharge region 60. Here, the support portion 63 may be a portion occupied by the support 50.

The discharge element 40 may have a certain cross-section in a direction perpendicular to the first direction x. The discharge element 40 may have a certain cross-section and extend in the first direction x. The discharge element 40 may include a protrusion pattern 400. The electric field formed between the discharge element 40 and the second discharge unit 62 may be strengthened through the protrusion pattern 400. The protrusion pattern 400 may induce the formation of a stable plasma electric field in the discharge element 40. The protrusion pattern 400 of the discharge element 40 may be formed symmetrically with respect to the center O. The protrusion pattern 400 of the discharge element 40 may have a plurality of protrusions disposed radially. The protrusion pattern 400 of the discharge element 40 may have the plurality of protrusions each having a sharp end disposed along the circumference of the discharge element 40. In an embodiment, for example, the protrusion pattern 400 of the discharge element 40 may have eight protrusions disposed radially. However, the shape of the discharge element 40 is not limited thereto. The cross-sectional shape of the discharge element 40 may vary as long as the discharge element 40 has a structure capable of inducing the formation of the stable plasma electric field in the discharge region 60. In an embodiment, for example, the discharge element 40 may have a circular cross-section without having the protrusion pattern 400.

In an embodiment, each of the discharge element 40 and the support 50 may be provided in a plurality. A plurality of supports 50 and a plurality of discharge elements 40 may be disposed with a certain relationship inside the reactor 20. Hereinafter, the arrangement relationship of the plurality of supports 50 and the plurality of discharge elements 40 will be described.

Referring again to FIGS. 1, 5, and 6, the air purifier 1 according to an embodiment may include the plurality of supports 50 and the plurality of discharge elements 40. In such an embodiment where the plurality of supports 50 and the plurality of discharge elements 40 are disposed in the hollow region 200 inside the reactor 20, the plurality of supports 50 and the plurality of discharge elements 40 may be disposed with a certain positional relationship with each other. In an embodiment, for example, the plurality of supports 50 and the plurality of discharge elements 40 may be alternately disposed in the first direction x in the hollow region 200 inside the reactor 20.

In an embodiment where the plurality of supports 50 are disposed in the hollow region 200 of the reactor 20, the plurality of supports 50 may be spaced apart from each other in the first direction x. The plurality of supports 50 may be spaced apart from each other in the first direction x in the hollow region 200 of the reactor 20 to respectively support the discharge elements 40 on opposing sides. The plurality of supports 50 may be spaced apart from each other in the first direction x in the hollow region 200 of the reactor 20 to efficiently act the plasma generated in the discharge region 60 on the catalyst 52 included in the support 50. The plurality of supports 50 may be spaced apart from each other in the first direction x in the hollow region 200 of the reactor 20 to efficiently control a proportion of purification of the polluted air Air₁ by the active specie of plasma and purification of polluted air Air₁ by catalyst in the entire air purification. In an embodiment, for example, the discharge element 40 may be a discharge tip applied to a dielectric barrier discharge structure or a corona discharge structure, but is not limited thereto.

The plurality of supports 50 may be spaced apart from each other at even (constant or regular) distances in the first direction x inside the reactor 20. A separation distance between two adjacent supports 50 among the plurality of supports 50 may correspond to a thickness of the discharge element 40 disposed between the two adjacent supports 50. The thickness of the discharge element 40 may be a length of the discharge element 40 having a certain cross-section and extending in the first direction x. The plurality of supports 50 may be spaced apart from each other by the thickness of the discharge element 40 inside the reactor 20. The discharge element 40 may be disposed between any two adjacent supports 50 among the plurality of supports 50. In other words, one support 50, the discharge element 40, and another support 50 may be disposed in this order inside the reactor 20. However, a way in which the plurality of supports 50 are disposed inside the reactor 20 is not limited to the above description. In an embodiment, for example, at least one support 50 among the plurality of supports 50 may be spaced apart from each other at a distance different from a distance at which the remaining supports 50 are spaced apart from each other in the first direction x.

In an embodiment where the plurality of discharge elements 40 are disposed in the hollow region 200 of the reactor 20, the plurality of discharge elements 40 may be spaced apart from each other in the first direction x. In such an embodiment where the plurality of discharge elements 40 are disposed in the hollow region 200, the plurality of discharge elements 40 may be spaced apart from each other in the first direction x to uniformly distribute positions where plasma is generated on the discharge region 60 within the reactor 20. In such an embodiment where the plurality of discharge elements 40 are disposed in the hollow region 200, the plurality of discharge elements 40 may be spaced apart from each other in the first direction x to uniformly distribute positions where plasma is intensively generated on the discharge region 60 within the reactor 20. In such an embodiment where the plurality of discharge elements 40 are disposed in the hollow region 200, the plurality of discharge elements 40 may be spaced apart from each other in the first direction x to efficiently act the plasma generated in the discharge region 60 on the catalyst 52 included in the adjacent support 50. In such an embodiment where the plurality of discharge elements 40 are disposed in the hollow region 200, the plurality of discharge elements 40 may be spaced apart from each other in the first direction x to efficiently control the proportion of purification of the polluted air Air₁ by the active specie of plasma and purification of polluted air Air₁ by the catalyst in the entire air purification.

In an embodiment, positions of the plurality of discharge elements 40 may be fixed on the first electrode unit 31. A relative position of each of the plurality of discharge elements 40 with respect to the first electrode unit 31 may be fixed at certain distances in the first direction x on the first electrode unit 31. The positions of the plurality of discharge elements 40 may be fixed on the first electrode unit 31 so that the plurality of discharge elements 40 may be spaced apart from each other at a certain distance in the first direction x in the hollow region 200 of the reactor 20. The positions of at least some of the plurality of discharge elements 40 may be fixed on the first electrode unit 31 by the plurality of supports 50 supporting the discharge elements 40 on both sides. At least some (e.g., at least two) of the plurality of discharge elements 40 may be supported on both opposing sides in the first direction x by the plurality of supports 50. In other words, at least some of the plurality of discharge elements 40 may be disposed in spaces between the plurality of adjacent supports 50.

At least some of the plurality of discharge elements 40 according to an embodiment may be disposed in the spaces between the plurality of adjacent supports 50. The spaces between the plurality of adjacent supports 50 may be spaces where the discharge elements 40 are disposed. The discharge elements 40 may be disposed in the spaces between the plurality of adjacent supports 50 in the discharge region 60 inside the reactor 20. The spaces between the plurality of adjacent supports 50 may be parts where the discharge elements 40 are disposed and plasma generation is concentrated. The spaces between the plurality of adjacent supports 50 may define the first discharge units 61. The first discharge units 61 may be spaces where at least some of the plurality of discharge elements 40 are disposed. The first discharge unit 61 may be the space where the discharge element 40 is disposed inside the discharge region 60. The first discharge unit 61 may be the space where the discharge element 40 generates the plasma. The first discharge unit 61 inside the discharge region 60 may be the space where the plasma generation is concentrated within the reactor 20 of the air purifier 1.

The discharge element 40 disposed on the first discharge unit 61 according to an embodiment may contact the plurality of adjacent supports 50 on both opposing sides thereof. The discharge element 40 disposed in the first discharge unit 61 may be supported by the plurality of adjacent supports 50 on both opposing sides. Movement of the discharge element 40 disposed in the first discharge unit 61 in the first direction x may be limited by the plurality of adjacent supports 50. In other words, a degree of freedom with which the discharge element 40 disposed in the first discharge unit 61 may move is limited by the plurality of supports 50 adjacent to the discharge elements 40 on both opposing sides, and thus, the movement of the discharge element 40 in the first direction x inside the reactor 20 may be limited. In other words, the plurality of adjacent supports 50 may fix the positions of the discharge elements 40 disposed in the spaces therebetween. Fixing the positions of the discharge elements 40 inside the reactor 20 may mean fixing the positions where the discharge elements 40 are coupled to the first electrode unit 31. The plurality of adjacent supports 50 may fix coupling positions of the discharge elements 40 disposed therebetween on the first electrode unit 31. The plurality of adjacent supports 50 may control positions of the discharge elements 40 disposed therebetween on the hollow region 200 within the reactor 20. In other words, the plurality of adjacent supports 50 may fix the positions of the discharge elements 40 disposed therebetween so that the discharge element 40 is disposed in the first discharge unit 61 on the hollow region 200 within the reactor 20 to form the plasma. When the positions where the discharge elements 40 are disposed within the reactor 20 are fixed, the generation of plasma may be concentrated in a certain region within the reactor 20. The plurality of supports 50 may function as spacers configured to adjust the distance between the discharge elements 40 inside the reactor 20. However, the arrangement relationship between the plurality of supports 50 and the discharge elements 40 is not limited to the above description.

In an embodiment, as described above, the discharge elements 40 are disposed in the spaces between the plurality of supports 50 disposed adjacently. In such an embodiment, the supports 50 may be disposed between the plurality of discharge elements 40 disposed adjacently. For example, the support 50 disposed on the first discharge unit 61 may contact the plurality of adjacent discharge elements 40 on both opposing sides. For example, the discharge elements 40 may contact both opposing sides of the support 50 disposed on the first discharge unit 61 in the first direction x.

In an embodiment where the discharge elements 40 are disposed adjacent to the support 50, the plasma generated by the discharge element 40 may well reach the support 50. In an embodiment where the discharge elements 40 are disposed adjacent to the support 50, the plasma generated by the discharge element 40 may well reach the support 50. In an embodiment where the discharge elements 40 are disposed adjacent to the support 50, the activation efficiency of the catalyst 52 coated along the surface of the support 50 may be high.

The discharge elements 40 may be respectively disposed adjacent to both opposing sides of the support 50 according to an embodiment in the first direction x. The discharge elements 40 may respectively contact both opposing sides of the support 50 in the first direction x. In an embodiment where the support 50 contacts the discharge element 40 on both opposing sides thereof, a contact area between the support 50 and the discharge element 40 may be large. In an embodiment where the support 50 contacts the discharge element 40 on both opposing sides, plasma generated by the discharge element 40 may be transmitted through both sides of the support 50. In an embodiment where the support 50 contacts the discharge element 40 on both opposing sides, the activation efficiency of the catalyst 52 coated along the surface of the support 50 may be high.

In an embodiment, the discharge elements 40 may be disposed in the spaces between the plurality of supports 50 disposed adjacent to each other, and simultaneously, the support 50 may be disposed between the plurality of discharge elements 40 disposed adjacent to each other. In other words, the plurality of supports 50 and the plurality of discharge elements 40 may be alternately disposed in the first direction x.

The plurality of supports 50 and the plurality of discharge elements 40 according to an embodiment may be alternately disposed within the reactor 20 and form the discharge region 60. The plurality of supports 50 and the plurality of discharge elements 40 may alternately contact each other in the first direction x. In an embodiment, for example, one support 50, one discharge element 40, another support 50, and another discharge element 40 may be disposed in contact with each other in order in the first direction x in at least a part of the discharge region 60. In an embodiment where the plurality of supports 50 and the plurality of discharge elements 40 alternately contact each other in the first direction x, the contact area between the plurality of supports 50 and the plurality of discharge elements 40 may be large. In an embodiment where the plurality of supports 50 and the plurality of discharge elements 40 alternately contact each other in the first direction x, the plasma generated by the plurality of discharge elements 40 may well reach the catalyst 52 coated along the surface of each of the plurality of supports 50. In an embodiment where the plurality of supports 50 and the plurality of discharge elements 40 alternately contact each other in the first direction x, a synergistic effect may occur in the purification of the polluted air Air₁ by plasma and the purification of the polluted air Air₁ by the catalyst inside the reactor 20. In an embodiment where the plurality of supports 50 and the plurality of discharge elements 40 alternately contact each other in the first direction x, the air purification efficiency of the air purifier 1 may be high. However, the arrangement of the plurality of supports 50 and the plurality of discharge elements 40 is not limited to the above description. The arrangement of the plurality of supports 50 and the plurality of discharge elements 40 may be configured in various ways as long as the plurality of supports 50 and the plurality of discharge elements 40 collectively form a structure capable of purifying the polluted air Air₁ by plasma and catalyst inside the reactor 20 and producing the synergy effect. In an embodiment, for example, the number of supports 50, the thickness of the supports 50 extending in the first direction x, the number of discharge elements 40, the thickness of the discharge elements 40 extending in the first direction x, an inner diameter of the reactor 20, etc. may be variously adjusted to increase air purification efficiency in the air purifier 1.

In an embodiment, where the plurality of supports 50 and the plurality of discharge elements 40 form the discharge region 60 inside the reactor 20, the discharge elements 40 may be respectively disposed at both opposing ends of the discharge region 60. In such an embodiment, the discharge elements 40 may be disposed at both opposing ends of the plurality of supports 50 and the plurality of discharge elements 40 disposed to alternately contact each other in the first direction x. In other words, the number of discharge elements 40 disposed in the discharge region 60 may be at least one more than the number of supports 50. In an embodiment, for example, four discharge elements 40 may be spaced apart within the discharge region 60 and three supports 50 may be disposed between the adjacent discharge elements 40. In an embodiment, referring to FIG. 6, any one discharge element 40 may be disposed on one side of the discharge region 60 opposite to a direction in which the polluted air Air₁ enters the air purifier 1, and another discharge element 40 may be disposed on the other side of the discharge region 60 opposite to the direction in which the air purifier 1 discharges the purified air Air₂ to the outside. In an embodiment where the plurality of discharge elements 40 and the plurality of supports 50 are alternately disposed inside the discharge region 60 and simultaneously the discharge elements 40 are respectively disposed at both opposing ends of the discharge region 60, the discharge elements 40 may be disposed on both opposing sides of the support 50 inside the discharge region 60. In an embodiment where the discharge elements 40 are disposed on both opposing sides of the support 50 inside the discharge region 60, the air purification efficiency of the air purifier 1 may be high.

The plasma generated by the discharge elements 40 in contact with the support 50 on both opposing sides in the first direction x may activate the catalyst relatively well. A space where the discharge element 40 in contact with the support 50 on both opposing sides in the first direction x is disposed within the discharge region 60 may be referred to as the first discharge unit 61. The first discharge unit 61 may be the space where the discharge element 40 in contact with the support 50 are disposed at both opposing sides so that plasma may activate the catalyst relatively well.

In an embodiment where the discharge elements 40 are respectively disposed at both opposing ends of the discharge region 60, the discharge elements 40 disposed at both opposing ends of the discharge region 60 may expose the other side opposite to one side disposed adjacent to the support 50 to the outside of the discharge region 60. Both opposing ends of the discharge region 60 may define the second discharge unit 62. The discharge element 40 disposed in the second discharge unit 62 may expose one side to the outside of the discharge region 60. The position of the discharge element 40 disposed in the second discharge unit 62 may be fixed to the first electrode unit 31. In an embodiment, for example, the position of the discharge element 40 disposed in the second discharge unit 62 may be fixed to the first electrode unit 31 by welding the discharge element 40 to the first electrode unit 31. In an embodiment, for example, the discharge element 40 disposed in the second discharge unit 62 is manufactured integrally with the first electrode unit 31 so that the position of the discharge element 40 may be fixed to the first electrode unit 31. In an embodiment, for example, the discharge element 40 disposed in the second discharge unit 62 is supported on at least one side by a coupling clip (not shown) coupled to the first electrode unit 31 so that the position of the discharge element 40 may be fixed to the first electrode unit 31. In an embodiment, for example, one side of both opposing sides of the discharge element 40 disposed in the second discharge unit 62 may contact the support 50 and the other side may contact the coupling clip (not shown). In an embodiment, for example, the position of the discharge element 40 disposed in the second discharge unit 62 may be fixed to the first electrode unit 31 by the support 50 and the coupling clip (not shown) respectively disposed on both opposing sides. However, the above description of the arrangement of the discharge element 40 and the support 50 is only an example and the invention is not limited thereto.

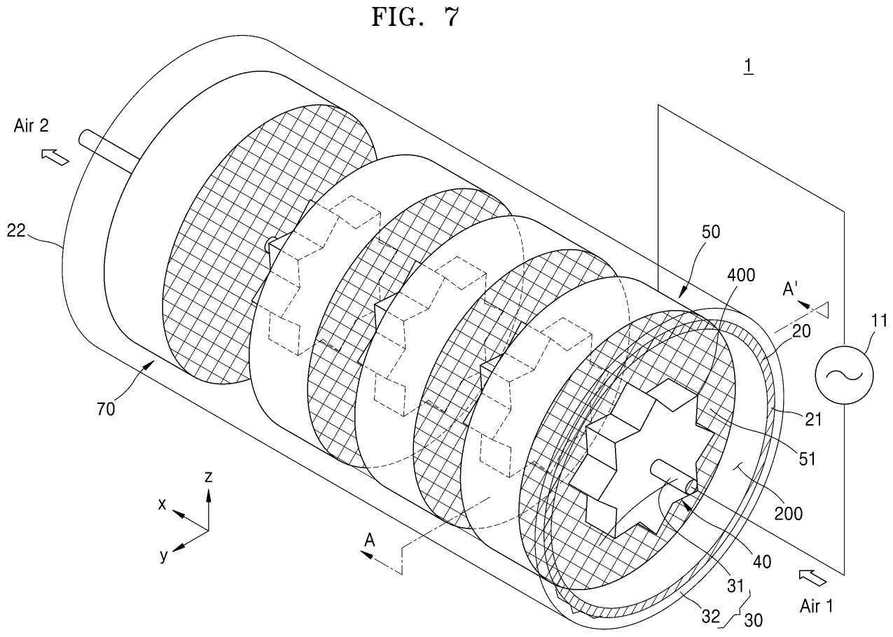

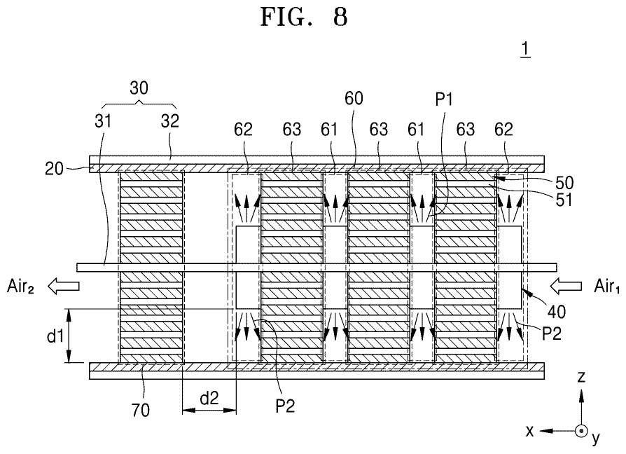

FIG. 7 is a perspective view of the air purifier 1 further including an external catalyst 70 according to an embodiment. FIG. 8 is a cross-sectional view of the air purifier 1 further including the external catalyst 70 according to an embodiment.

Referring to FIGS. 6 to 8, the air purifier 1 according to an embodiment may further include the external catalyst 70. The external catalyst 70 may be disposed outside the discharge region 60. The catalyst 52 configured to purify the polluted air Air₁ may be coated along a surface of the external catalyst 70. The external catalyst 70 may be provided with the plurality of through holes 51 defined therein to extend in the first direction x. The external catalyst 70 may have a honeycomb structure. The external catalyst 70 may have substantially the same structure as the support 50 described in FIGS. 1 to 6, but may be distinguished from the support 50 in that the external catalyst 70 is disposed outside the discharge region 60. In other words, the support 50 is in-plasma catalysis (IPC) disposed inside the discharge region 60, and the external catalyst 70 is post-plasma catalysis (PPC) disposed outside the discharge region 60 so that the external catalyst 70 may be distinguished from the support 50. However, a relationship between the external catalyst 70 and the discharge region 60 is not limited to the above description.

The external catalyst 70 according to an embodiment may be spaced apart from the discharge region 60 by a certain distance and disposed outside the discharge region 60. The external catalyst 70 may be spaced apart from the discharge element 40 in the first direction x. The external catalyst 70 may not contact the discharge element 40 disposed in the second discharge unit 62 of the discharge region 60. The external catalyst 70 may not support the discharge element 40. The external catalyst 70 may be distinguished from the support 50 in that the external catalyst 70 may not function to support the discharge element 40 inside the reactor 20.

However, a positional relationship between the external catalyst 70 and the discharge element 40 is not limited to the above description. In an embodiment, for example, the external catalyst 70 may contact the discharge element 40 disposed in the second discharge unit 62 and support the discharge element 40 outside the discharge region 60. In such an embodiment, when defining the discharge region 60, among the plurality of discharge elements 40, a space between one discharge element 40 disposed closest to a region where the polluted air Air₁ flows into the reactor 20 with respect to the first direction x inside the reactor 20 and another discharge element 40 disposed closest to a region where the purified air Air₂ is discharged from the reactor 20 may be defined as the discharge region 60. In other words, the discharge region 60 may be defined according to a position at which the discharge element 40 is disposed with respect to the first direction x inside the reactor 20. However, a method of defining the discharge region 60 inside the reactor 20 is not limited to the above description. In an embodiment, for example, a region where a plasma discharge is concentrated inside the reactor 20 may be defined as the discharge region 60.

The external catalyst 70 and the support 50 according to an embodiment may be similar to each other in that the external catalyst 70 and the support 50 include the catalyst 52 including the plurality of through holes 51 each extending in the first direction x, surrounding the first electrode unit 31, disposed in the hollow region 200 of the support 50, and coated along the surface of the support 50. The criterion for distinguishing the external catalyst 70 from the support 50 may depend on whether to be disposed outside the discharge region 60. In other words, even a separate configuration having the same shape may be referred to as the external catalyst 70 when disposed outside the discharge region 60, and may be referred to as the support 50 when disposed inside the discharge region 60. However, the shape of the external catalyst 70 is not limited to the above description, and the external catalyst 70 may have a shape different from that of the support 50.

The external catalyst 70 may be disposed at a rear end of the discharge region 60. The rear end of the discharge region 60 may be one end of the discharge region 60 that faces a direction in which the purified air Air₂ is discharged to the outside of the air purifier 1. In other words, a region where the external catalyst 70 is disposed may be a region of one end of both opposing ends of the discharge region 60 adjacent to a region where the purified air Air₂ is discharged from the inside of the reactor 20. Air flowing in the first direction x through the hollow region 200 of the reactor 20 inside the air purifier 1 may pass through the discharge region 60 and reach the external catalyst 70. The external catalyst 70 may purify the polluted air Air₁ that is unpurified on the discharge region 60. However, the region where the external catalyst 70 is disposed and the function of the external catalyst 70 are not limited to the above description.

In a case, where the external catalyst 70 is disposed excessively spaced from the discharge region 60 in the first direction x, the plasma generated by the second discharge unit 62 may not reach the external catalyst 70. In other words, when the external catalyst 70 is disposed excessively spaced apart from the second discharge unit 62 of the discharge region 60, the plasma generated by the discharge element 40 may not reach the external catalyst 70. When the plasma generated by the discharge element 40 does not reach the external catalyst 70, the activation efficiency of the external catalyst 70 may decrease.

The external catalyst 70 according to an embodiment may be disposed adjacent to one end of the discharge region 60. The external catalyst 70 may be disposed adjacent to the second discharge unit 62. In such an embodiment where the external catalyst 70 is disposed adjacent to the second discharge unit 62, the plasma generated by the second discharge unit 62 may reach the external catalyst 70. In other words, when the external catalyst 70 is disposed adjacent to the second discharge unit 62, the plasma generated by the discharge element 40 disposed in the second discharge unit 62 may reach the external catalyst 70.

A separation distance between the external catalyst 70 and the discharge region 60 according to an embodiment may be a certain distance or less. In other words, the distance between the external catalyst 70 and the second discharge unit 62 may be the certain distance or less. More specifically, the distance between the external catalyst 70 and the second discharge unit 62 may be a distance or less at which the plasma generated by the discharge element 40 may move in a direction beyond the discharge region 60. However, the distance between the external catalyst 70 and the discharge region 60 is not limited to the above description.

In an embodiment, the distance at which the plasma generated by the discharge element 40 may move may be controlled by a distance between the discharge element 40 and the reactor 20. In other words, a distance at which a plasma emitted by the discharge element 40 may move may be determined by a distance between one end of the discharge element 40 and the reactor 20. However, the distance that the plasma emitted by the discharge element 40 may move is not limited to the above description, and may be determined by various factors such as a length of the discharge element 40 extending in the first direction x, a diameter of the first electrode unit 31, an arrangement of the electrode unit 32, a shape of the support 50, etc.

Referring back to FIGS. 5 to 8, the discharge element 40 according to an embodiment may be spaced apart from the reactor 20 by a first distance d1. One end of the discharge element 40 may be spaced apart from the reactor 20 by the first distance d1. The external catalyst 70 may be spaced apart from the discharge region 60 by a second distance d2. The external catalyst 70 may be spaced apart from the second discharge unit 62 by the second distance d2. The external catalyst 70 may be spaced apart from the discharge element 40 disposed in the second discharge unit 62 by the second distance d2.

The second distance d2 according to an embodiment may be 120% or less of the first distance d1. The second distance d2 may be 100% or less of the first distance d1. In an embodiment, for example, when the second distance d2 is smaller than the first distance d1, the distance between the second discharge part 62 and the external catalyst 70 may be shorter than the distance between the discharge element 40 and the reactor 20 disposed in the second discharge part 62. The external catalyst 70 may be within a certain distance from the second discharge unit 62 such that activation of the external catalyst 70 by the plasma may be induced. However, the arrangement of the discharge element 40 and the external catalyst 70 is not limited to the above description.

In an embodiment, the air purifier 1 may include a plasma reaction module. In an embodiment, the reactor 20, the first electrode unit 31, the plurality of supports 50, the plurality of discharge elements 40, and the second electrode unit 32 described above may act as or collectively define a plasma reaction module.

The plasma reaction module according to an embodiment may use one or more discharge methods among dielectric barrier discharge, corona discharge, arc discharge, microwave induced discharge, and radio frequency discharge. Hereinafter, the air purifier 1 including the plasma reaction module is described, but any repetitive detailed descriptions of the same or like elements as those described above will be omitted.

FIG. 9A is a front view of the plasma reaction module 10a according to an embodiment, FIG. 9B is a front view of the plasma reaction module 10b according to an embodiment, FIG. 9C is a front view of the plasma reaction module 10c according to an embodiment, FIG. 9D is a front view of the plasma reaction module 10d according to an embodiment,

Referring to FIGS. 9A to 9D, the reactor 20 according to an embodiment may have one or more cross-sections of a circular shape or polygonal shape. The second electrode unit 32 may surround the reactor 20. The second electrode unit 32 may surround the reactor 20 and have the same cross-section as the reactor 20. The plasma reaction module may generally have a cross-sections of a circular shape or polygonal shape. As the plasma reaction module has one or more cross sections of the circular shape or polygonal shape, the plurality of reactors 20 may be integrated and disposed adjacent to each other. Accordingly, the cross-sectional area of the reactor 20 through which the polluted air Air₁ passes through may be increased, and the entire air purifier 1 may be miniaturized.

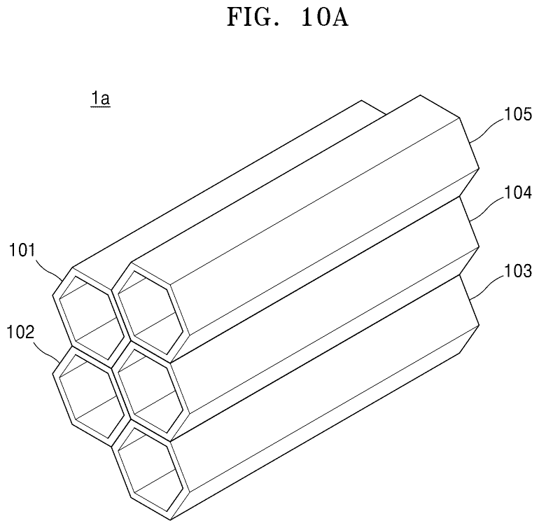

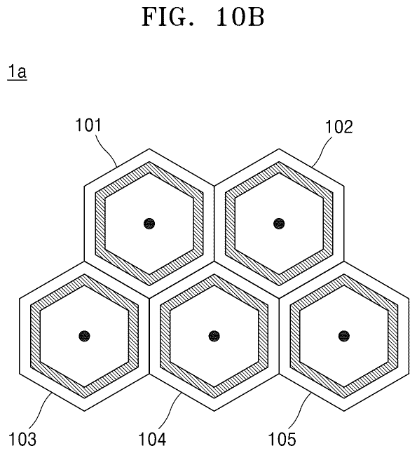

FIG. 10A is a perspective view of an air purifier 1a including the plurality of plasma reaction modules according to an embodiment. FIG. 10B is a front view of the air purifier 1a including the plurality of plasma reaction modules according to an embodiment.

Referring to FIGS. 10A and 10B, the air purifier 1a may include the plurality of plasma reaction modules according to an embodiment. For example, the plurality of plasma reaction modules may include five plasma reaction modules. In an embodiment, for example, the plurality of plasma reaction modules may include a first plasma reaction module 101 to a fifth plasma reaction module 105. However, the number of plasma reaction modules is not limited thereto, and the number of plasma reaction modules may be adjusted to two or more.

The first to fifth plasma reaction modules 101 to 105 according to an embodiment may each have one or more cross sections of a circular shape or a polygonal shape. In an embodiment, for example, the first to fifth plasma reaction modules 101 to 105 may each have a hexagonal cross-section. The first to fifth plasma reaction modules 101 to 105 may each have the hexagonal cross-section and be stacked adjacent to each other. Accordingly, a cross-sectional area of the reactor 20 through which the polluted air Air₁ passes may increase. Accordingly, a processing capacity of the air purifier 1a capable of processing the polluted air Air₁ may increase.

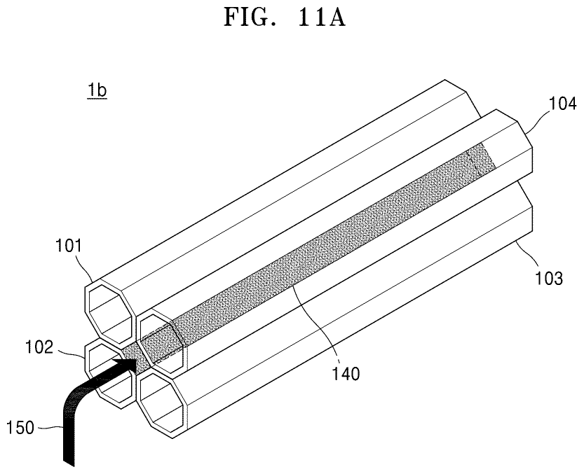

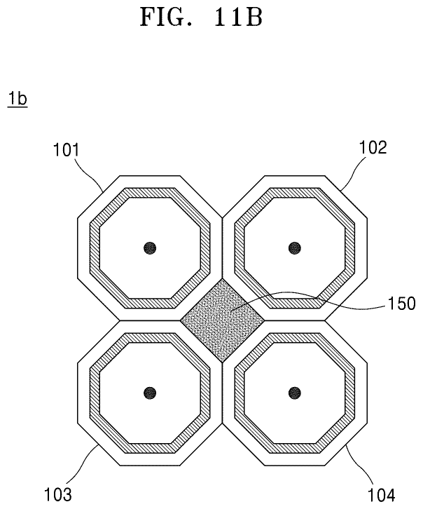

FIG. 11A is a perspective view of an air purifier 1b including a cooling flow path 140 according to an embodiment, and FIG. 11B is a front view of the air purifier 1b including the cooling flow path 140 according to an embodiment.

Referring to FIGS. 11A and 11B, the purifier 1b may include the plurality of plasma reaction according to an embodiment. In an embodiment, for example, the plurality of plasma reaction modules may include four plasma reaction modules. In an embodiment, for example, the plurality of plasma reaction modules may include a first plasma reaction module 101 to a fourth plasma reaction module 104.

The first plasma reaction module 101 to the fourth plasma reaction module 104 according to an embodiment may each have one or more cross sections of a circular shape or a polygonal shape. In an embodiment, for example, the first plasma reaction module 101 to the fourth plasma reaction module 104 may each have an octagonal cross-section. The first to fourth plasma reaction modules 101 to 104 may each have the octagonal cross-section such that the first to fourth plasma reaction modules 101 to 104 may be stacked adjacent to each other.

A certain separation space may be formed between the first plasma reaction module 101 to the fourth plasma reaction module 104 according to an embodiment. The cooling flow path 140 may be formed in the certain separation space. The cooling flow path 140 may extend in one direction in which the first plasma reaction module 101 to the fourth plasma reaction module 104 extend. The cooling water 150 may move in one direction in the cooling flow path 140. The cooling water 150 may be any fluid material, for example, water, capable of absorbing heat generated from the first plasma reaction module 101 to the fourth plasma reaction module 104. The cooling flow path 140 may be disposed between the plurality of plasma reaction modules, and the cooling water 150 moves along the cooling flow path 140, and thus, the air purifier 1b may discharge heat generated in a process of forming a discharge plasma to the outside. Accordingly, the efficiency of the air purifier 1b may be increased.

An air purifier according to an embodiment may purify polluted air containing organic compounds by using activated species generated in plasma to act on a catalyst.

An air purifier according to an embodiment may provide an air purifier with increased activation efficiency of a catalyst.

An air purifier according to an embodiment may provide an air purifier capable of purifying a large flow of polluted air with high efficiency.

The invention should not be construed as being limited to the embodiments set forth herein. Rather, these embodiments are provided so that this disclosure will be thorough and complete and will fully convey the concept of the invention to those skilled in the art.

While the invention has been particularly shown and described with reference to embodiments thereof, it will be understood by those of ordinary skill in the art that various changes in form and details may be made therein without departing from the spirit or scope of the invention as defined by the following claims.

What is claimed is:

1. An air purifier comprising:
a reactor comprising a hollow region extending in a first direction;
a first electrode unit extending in the first direction and disposed in the hollow region;
a plurality of supports disposed in the hollow region to surround the first electrode unit, and spaced apart from each other in the first direction, wherein a plurality of through holes extending in the first direction is defined in each of the plurality of supports;
a plurality of discharge elements coupled to the first electrode unit and disposed in the hollow region, wherein at least one of the plurality of discharge elements is disposed in a first discharge unit defined by a space between the plurality of supports; and
a second electrode unit spaced apart from the plurality of discharge elements by a certain distance on a plane perpendicular to the first direction to surround the plurality of discharge elements.

2. The air purifier of claim 1, wherein each of the plurality of supports includes a catalyst coated along a surface thereof.

3. The air purifier of claim 2, wherein one of the plurality of discharge elements is disposed between two adjacent supports among the plurality of supports.

4. The air purifier of claim 3, wherein the two adjacent supports fix a coupling position of the one of the plurality of discharge elements disposed therebetween on the first electrode unit.

5. The air purifier of claim 4, wherein the plurality of supports and the plurality of discharge elements are alternately disposed in the first direction.

6. The air purifier of claim 5, wherein the plurality of supports and the plurality of discharge elements alternately contact each other in the first direction.

7. The air purifier of claim 1, wherein the reactor includes a dielectric material.

8. The air purifier of claim 7, wherein the second electrode unit surrounds the reactor.

9. The air purifier of claim 8, wherein the discharge element includes a protrusion pattern protruding towards the second electrode unit.

10. The air purifier of claim 9, wherein each of the plurality of supports has a honeycomb structure.

11. The air purifier of claim 1, wherein
the plurality of discharge elements and the plurality of supports form a discharge region within the reactor, and
one of the plurality of discharge elements is disposed in each of the second discharge units defined by both opposing ends of the plurality of discharge regions.

12. The air purifier of claim 11, wherein the one of the plurality of discharge elements disposed in each of the second discharge units contacts an adjacent support.

13. The air purifier of claim 12, wherein a position of the one of the plurality of discharge elements disposed in each of the second discharge units is fixed to the first electrode unit.

14. The air purifier of claim 13, further comprising:
an external catalyst surrounding the first electrode unit and disposed outside the discharge region,
wherein the external catalyst is disposed at a rear end of the discharge region.

15. The air purifier of claim 14, wherein the external catalyst is disposed adjacent to one of the second discharge units.

16. The air purifier of claim 15, wherein a distance between the external catalyst and one of the second discharge units is 120% or less of a distance between the reactor and the discharge element disposed in the second discharge unit.

17. The air purifier of claim 16, wherein the distance between the external catalyst and one of the second discharge units is 100% or less of the distance between the reactor and the discharge element disposed in the second discharge unit.

18. The air purifier of claim 1, wherein the reactor has a cross-section of a circular shape or a polygonal shape.

19. The air purifier of claim 18, wherein
the reactor, the first electrode unit, the plurality of supports, the plurality of discharge elements, and the second electrode unit collectively define a plasma reaction module,
the plasma reaction module is provided in plural, and
a plurality of reactors respectively included in a plurality of plasma reaction modules are disposed to be adjacent to each other.

20. The air purifier of claim 19, further comprising:
a cooling flow path disposed between the plurality of reactors respectively included in the plurality of plasma reaction modules; and
cooling water provided to move along the cooling flow path.
